(19) United States
(12) Patent Application Publication
(10) Pub. No.: US 2023/0000984 A1
(43) Pub. Date: Jan. 5, 2023

(54) BIOCOMPATIBLE PHOTOTHERMAL COMPOSITION FOR TREATMENT OF CANCER AND SKIN DISEASES

(71) Applicant: SEOUL NATIONAL UNIVERSITY R&DB FOUNDATION, Seoul (KR)

(72) Inventors: Yu-Kyoung OH, Seoul (KR); Seungbeom Ko, Jeju-si Jeju-do (KR)

(73) Assignee: SEOUL NATIONAL UNIVERSITY R&DB FOUNDATION, Seoul (KR)

(21) Appl. No.: 17/821,471

(22) Filed: Aug. 22, 2022

Related U.S. Application Data
(63) Continuation of application No. 16/475,959, filed on Aug. 6, 2019.

(30) Foreign Application Priority Data
Jan. 4, 2017 (KR) 10-2017-0001171

(51) Int. Cl. A61K 41/00; A61K 33/244; A61K 31/235; A61K 33/06; A61K 33/24; A61K 33/26; A61K 33/30; A61K 33/32

(52) U.S. Cl. CPC A61K 41/0052; A61K 33/244; A61K 31/235; A61K 33/06; A61K 33/24; A61K 33/26; A61K 33/30; A61K 33/32

(57) ABSTRACT

The present invention relates to a biocompatible photothermal composition that can be used in various fields including the treatment of cancer and skin diseases.

### CROSS REFERENCE TO RELATED APPLICATIONS

This application is a continuation of U.S. patent application Ser. No. 16/475,959, filed Aug. 6, 2019, which is 371 of PCT/KR2018/000063, filed Jan. 2, 2018 which claims the benefit of Korean Patent Application No. 10-2017-0001171, filed Jan. 4, 2017, the contents of each of which are incorporated herein by reference.

### BACKGROUND OF THE INVENTION

### Field of the Invention

The present invention relates to a biocompatible photo thermal composition that can be used in various fields including the treatment of cancer and skin diseases.

### Description of the Related Art

Cancer, which is caused by various reasons including stress and pollution, is the leading cause of death of modern people. Cancer is caused by gene mutation in normal cells. Cancer indicates a malignant tumor among tumors which do not follow normal path of cell differentiation, cell growth and cell apoptosis. Methods of treating cancer include surgical operation, chemotherapy, and radiotherapy.

In the case of chemotherapy, a drug is administered systemically, and the drug not only kills cancer cells but also spreads to normal tissues to cause toxicity in normal cells as well. Accordingly, serious side effects such as gastrointestinal side effects, thrombocytopenia and hair loss are caused.

Chemotherapy based anticancer treatment is limited in the case of tumors resistant to chemotherapy by expressing p-glycoproteins.

In particular, a solid tumor has heterogeneity in tumor tissue, indicating that both the tumor cells having sensibility to chemotherapeutic agents and the other tumor cells showing resistance to chemotherapeutic agents exist together in the tissue so that the elimination of the resistant tumor cells alone is very difficult even after the administration of chemotherapeutic agents.

To solve the problem above, it is necessary to develop a novel anticancer agent based on a novel anticancer mechanism which can be applied locally to tumor tissues and overcome different sensibility among tumor cells. Thus, studies reflecting such necessity have been actively going on.

In particular, photothermal therapy is one of the most popular anticancer treatment methods. This method uses the weakness of cancer cells on heat, compared with normal cells, so that a photoresponsive material is located in a local area where cancer cells are located and then heat is generated by a stimulus given from outside to kill cancer cells selectively.

For example, methods using gold nanoparticles, nanoporous silica or carbon nanotubes as a photoresponsive material or using organic polymer nanoparticles have been developed (Patent Reference 1, Korean Patent Publication No. 10-2012-0107686).

The present inventors have been tried to develop an anticancer agent that can overcome side effects according to systemic administration and difficulty in eliminating tumor cells having resistance to chemotherapeutic agents. In the course of our study, the present inventors developed a biocompatible photothermal composition capable of acting selectively on a local site, and confirmed its photothermal effect and photothermal therapeutic effect on cancer cells, leading to the completion of the present invention.

The present inventors also confirmed the antimicrobial effect of the said photothermal composition and thereafter applied the composition to the treatment of skin disease and extended the use of the composition for accelerating absorption of functional materials for cosmetics.

### SUMMARY OF THE INVENTION

It is an object of the present invention to provide a photothermal composition comprising a metal salt and a benzene ring compound derivative containing two or more hydroxy groups.

To achieve the above object, the present invention provides a photothermal composition comprising a metal salt; and a catechol derivative.

### Advantageous Effect

The composition of the present invention displays a remarkable effect on photothermal therapy since the temperature of the applied area can be raised at least 50° C. by near infrared ray irradiation, after the injection. The composition can be combined with a biocompatible material to have biocompatibility and can act selectively on a local site to minimize side effects. The composition also has an effect of continuous photothermal treatment because it is present in the administration site for a few days after injection. Therefore, the composition of the present invention can be used for anticancer treatment. In addition, the composition of the present invention exhibits an antibacterial effect, so that it can be used for treating skin disease and for increasing skin absorption of functional materials for cosmetics through photothermal effect.

### BRIEF DESCRIPTION OF THE DRAWINGS

FIG. 1 is a graph illustrating the temperature changes according to the irradiation of infrared ray laser to catechol or a coordination complex of catechol and iron ions.

FIG. 2 is a graph illustrating the temperature changes according to the irradiation of infrared ray laser to dopamine or a coordination complex of dopamine and iron ions.

FIG. 3 is a graph illustrating the temperature changes according to the irradiation of infrared ray laser to epigallocatechin gallate or a coordination complex of epigallocatechin and iron ions.

FIG. 4 is a graph illustrating the temperature changes according to the irradiation of infrared ray laser to gallic acid or a coordination complex of gallic acid and iron ions.

FIG. 5 is a graph illustrating the temperature changes according to the irradiation of infrared ray laser to tannic acid or a coordination complex of tannic acid and iron ions.

FIG. 6 is a graph illustrating the cell survival rate in photothermal therapy using a coordination complex of catechol and iron ions.

FIG. 7 is a graph illustrating the cell survival rate in photothermal therapy using a coordination complex of dopamine and iron ions.

FIG. 8 is a graph illustrating the cell survival rate in photothermal therapy using a coordination complex of epigallocatechin gallate and iron ions.

FIG. 9 is a graph illustrating the cell survival rate in photothermal therapy using a coordination complex of gallic acid and iron ions.

FIG. 10 is a graph illustrating the cell survival rate in photothermal therapy using a coordination complex of tannic acid and iron ions.

FIG. 11 is a diagram illustrating the synthesis process of a hyaluronic acid-gallic acid conjugate according to an example of the present invention. Herein, HA-GA indicates the hyaluronic acid-gallic acid conjugate.

FIG. 12 is a set of photographs illustrating the generation of hydrogel when the hyaluronic acid-gallic acid conjugate in liquid phase is mixed with liquid iron chloride. Herein, GA indicates gallic acid and HA-GA indicates the hyaluronic acid-gallic acid conjugate.

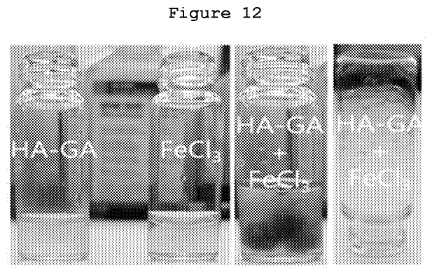

FIG. 13 is a graph illustrating the time-dependent swelling of the hydrogel formed by the hyaluronic acid-gallic acid conjugate and iron ions.

FIG. 14 is a graph illustrating the hertz-dependent viscosity of the hydrogel formed by the hyaluronic acid-gallic acid conjugate and iron ions.

FIG. 15 is a graph illustrating the hertz-dependent viscoelasticity of the hydrogel formed by the hyaluronic acid-gallic acid conjugate and iron ions.

FIG. 16 is a set of thermograms of the hydrogel formed by the hyaluronic acid-gallic acid conjugate and iron ions according to the irradiation of infrared ray laser.

FIG. 17 is a graph illustrating the temperature change of the hydrogel formed by the hyaluronic acid-gallic acid conjugate and iron ions according to the irradiation of infrared ray laser.

FIG. 18 is a graph illustrating the cell viability according to photothermal therapy using the hydrogel formed by the hyaluronic acid-gallic acid conjugate and iron ions.

FIG. 19 is a set of live cell staining images illustrating the cell viability according to photothermal therapy using the hydrogel formed by the hyaluronic acid-gallic acid conjugate and iron ions.

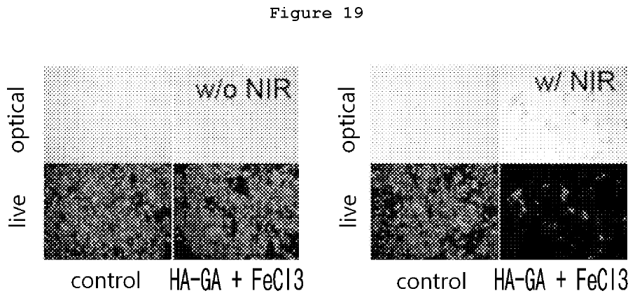

FIG. 20 is a set of photographs illustrating the hydrogel formed under the mouse subcutis by the hyaluronic acid-gallic acid conjugate and iron ions.

FIG. 21 is a graph illustrating the sustainability of the photothermal effect of the hyaluronic acid-gallic acid conjugate under the mouse subcutis.

FIG. 22 is a graph illustrating the sustainability of the photothermal effect of the hydrogel formed by the hyaluronic acid-gallic acid conjugate and iron ions.

FIG. 23 is a graph illustrating the time-dependent changes in tumor size according to photothermal therapy using the hydrogel formed by the hyaluronic acid-gallic acid conjugate and iron ions.

FIG. 24 is a photograph illustrating the mixture of hydrogel and a coordination complex of gallic acid and iron.

FIG. 25 is a table illustrating the maximum temperature of the mixture of hydrogel and a coordination complex of gallic acid and iron according to the laser intensity and distance.

FIG. 26 is a set of photographs illustrating the antibacterial effect of a coordination complex of dopamine and iron.

FIG. 27 is a set of photographs illustrating the antibacterial effect of a coordination complex of epigallocatechin and iron.

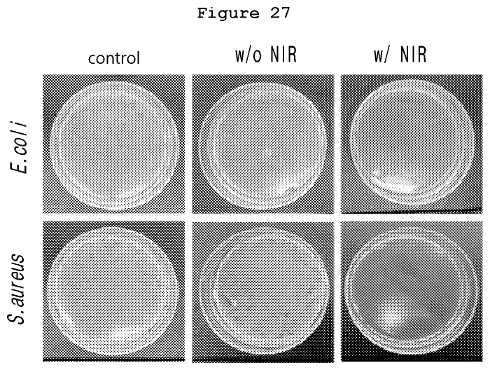

FIG. 28 is a set of photographs illustrating the antibacterial effect of a coordination complex of gallic acid and iron.

FIG. 29 is a set of photographs illustrating the antibacterial effect of a coordination complex of tannic acid and iron.

### DESCRIPTION OF THE PREFERRED EMBODIMENTS

Hereinafter, the present invention is described in detail.

The present invention provides a photothermal composition comprising a metal salt; and a catechol derivative.

Herein, the photothermal composition is characterized by comprising tannic acid or the compound represented by formula 1 below as the catechol derivative.

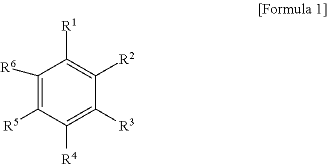

In formula 1 above,

R¹ and R² are —OH;

R³ is —H, —OH, —CN, —NO₂, halogen, —COOM, amine C₁₋₅ straight or branched alkyl, C₁₋₅ straight or branched alkyl, C₁₋₅ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S, and R³ is linked together with R⁴ to form unsubstituted or substituted C₆₋₁₀ aryl,

wherein, M is —H, C₁₋₅ straight or branched alkyl or epigallocatechinyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₅ straight or branched alkyl and C₁₋₅ straight or branched alkoxy;

R⁴ is —H, —OH, —CN, —NO₂, halogen, —COOM, —CH(OH)—CH₂—NHA¹, amine C₁₋₅ straight or branched alkyl, C₁₋₅ straight or branched alkyl, C₁₋₅ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S, and R⁴ is linked together with R⁵ to form unsubstituted or substituted C₆₋₁₀ aryl,

wherein, M is —H, C₁₋₅ straight or branched alkyl or epigallocatechinyl,

A¹ is —H or C₁₋₅ straight or branched alkyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₅ straight or branched alkyl and C₁₋₅ straight or branched alkoxy;

R⁵ is —H, —OH, —CN, —NO₂, halogen, —COOM, amine C₁₋₅ straight or branched alkyl, C₁₋₅ straight or branched alkyl, C₁₋₅ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S, and R⁵ is linked together with R⁶ to form unsubstituted or substituted C₆₋₁₀ aryl,

wherein, M is —H, C₁₋₅ straight or branched alkyl or epigallocatechinyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₅ straight or branched alkyl and C₁₋₅ straight or branched alkoxy; and

R⁶ is —H, —OH, —CN, —NO₂, halogen, —COOM, amine C₁₋₅ straight or branched alkyl, C₁₋₅ straight or branched alkyl, C₁₋₅ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S,

wherein, M is —H, C₁₋₅ straight or branched alkyl or epigallocatechinyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₅ straight or branched alkyl and C₁₋₅ straight or branched alkoxy.

Preferably,

R¹ and R² are —OH;

R³ is —H, —OH, —CN, —NO₂, halogen, —COOM, amine C₁₋₃ straight or branched alkyl, C₁₋₃ straight or branched alkyl, C₁₋₃ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S, and R³ is linked together with R⁴ to form unsubstituted or substituted C₆₋₁₀ aryl,

wherein, M is —H, C₁₋₅ straight or branched alkyl or epigallocatechinyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₃ straight or branched alkyl and C₁₋₃ straight or branched alkoxy;

R⁴ is —H, —OH, —CN, —NO₂, halogen, —COOM, —CH(OH)—CH₂—NHA¹, amine C₁₋₃ straight or branched alkyl, C₁₋₃ straight or branched alkyl, C₁₋₃ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S, and R⁴ is linked together with R⁵ to form unsubstituted or substituted C₆₋₁₀ aryl,

wherein, M is —H, C₁₋₅ straight or branched alkyl or epigallocatechinyl,

A¹ is —H or C₁₋₅ straight or branched alkyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₃ straight or branched alkyl and C₁₋₃ straight or branched alkoxy;

R⁵ is —H, —OH, —CN, —NO₂, halogen, —COOM, amine C₁₋₃ straight or branched alkyl, C₁₋₃ straight or branched alkyl, C₁₋₃ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S. and R⁵ is linked together with R⁶ to form unsubstituted or substituted C₆₋₁₀ aryl,

wherein, M is —H, C₁₋₅ straight or branched alkyl or epigallocatechinyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₃ straight or branched alkyl and C₁₋₃ straight or branched alkoxy; and

R⁶ is —H, —OH, —CN, —NO₂, halogen, —COOM, amine C₁₋₃ straight or branched alkyl, C₁₋₃ straight or branched alkyl, C₁₋₃ straight or branched alkoxy, unsubstituted or substituted C₆₋₁₀ aryl, unsubstituted or substituted C₃₋₁₀ cycloalkyl, unsubstituted or substituted 5-10 membered heteroaryl containing one or more hetero atoms selected from the group consisting of N, O and S, or unsubstituted or substituted 5-10 membered heterocycloalkyl containing one or more hetero atoms selected from the group consisting of N, O and S,

wherein, M is —H, C₁₋₅ straight or branched alkyl or epigallocatechinyl,

the substituted C₆₋₁₀ aryl, C₃₋₁₀ cycloalkyl, 5-10 membered heteroaryl and 5-10 membered heterocycloalkyl are independently C₆₋₁₀ aryl, C₃₋₁₀ cycloalkyl, 5-10 membered heteroaryl and 5-10 membered heterocycloalkyl substituted with one or more substituents selected from the group consisting of —OH, C₁₋₃ straight or branched alkyl and C₁₋₃ straight or branched alkoxy.

In addition, the metal salt is a lanthanide metal salt or a transition metal salt. Examples of the metal of the lanthanide metal salt are cerium (Ce), europium (Eu), gadolinium (Gd) and terbium (Tb). Examples of the metal of the transition metal salt are aluminum (Al), vanadium (V), manganese (Mn), iron (Fe), zinc (Zn), zirconium (Zr), molybdenum (Mo), ruthenium (Ru) and rhodium (Rh).

Further, the metal ion of the metal salt characteristically forms a complex with the catechol derivative above.

The catechol derivative is characteristically linked to a biocompatible substance through covalent bond.

Herein, the biocompatible substance is exemplified by hyaluronic acid, methylcellulose, carboxymethylcellulose, hydroxypropylmethylcellulose, alginate, chitosan, gelatin and collagen.

For example, the compound represented by formula 1 can be fixed with hyaluronic acid which is one of biocompatible substances, as shown in formula 2 below.

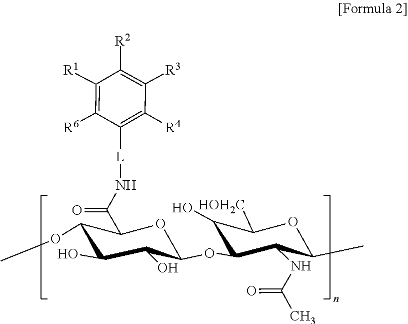

Herein, L is C₁₋₁₀ straight or branched alkylene or

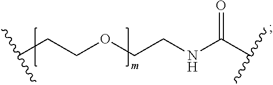

m is an integer selected from 1-5; and

n is an integer selected from 1-1000.

R¹, R², R³, R⁴ and R⁶ are independently as defined in formula 1 above.

When hyaluronic acid is used as a biocompatible substance to combine with a metal salt and a catechol derivative, the resulting complex is preferably in the form of hydrogel.

In general, hydrogel is hard to be injected in the form of injections. However, when a catechol derivative in which a metal salt and hyaluronic acid are linked together is injected hypodermically using a dual syringe, it can form hydrogel under the subcutis, suggesting that it can be applied locally for selective photothermal therapy.

Further, a complex in the form of hydrogel can be formulated as a patch, a depot or an external preparation. When a drug is incorporated in the preparation of hydrogel, the prepared sustained-release hydrogel containing the drug can be used for prolonged release of a therapeutic drug.

The photothermal composition of the present invention demonstrated sustainability of photothermal effect at 50° C. or higher for at least 6 days by infrared ray irradiation after the injection of the composition. Thus, long term photothermal therapy can be achieved by a single administration of the composition.

The photothermal composition of the present invention can be administered by a pathway selected from the group consisting of intravenous injection, intraperitoneal injection, intramuscular injection, intracranial injection, intratumoral injection, intraepithelial injection, transdermal delivery, esophageal administration, abdominal administration, intraarterial injection, intraarticular injection and intraoral administration.

In addition, the photothermal composition of the present invention is characteristically used for treating cancer, and at this time the cancer can be a solid tumor or a blood cancer.

The solid tumor above is specifically exemplified by brain tumor, benign astrocytoma, malignant astrocytoma, pituitary adenoma, meningioma, brain lymphoma, oligodendroglioma, intracranial carcinoma, ependymoma, brain stem tumor, head and neck cancer, laryngeal cancer, oropharyngeal cancer, nasal cavity cancer, nasopharyngeal cancer, salivary gland cancer, hypopharyngeal cancer, thyroid cancer, oral cancer, thoracic tumor, small cell lung cancer, non-small cell lung cancer, thymic carcinoma, mediastinal tumor, esophageal cancer, breast cancer, male breast cancer, abdominal tumor, stomach cancer, liver cancer, gallbladder cancer, bile duct cancer, pancreatic cancer, small bowel cancer, colon cancer, anal cancer, bladder cancer, kidney cancer, male genital tumor, penile cancer, prostate cancer, female genital tumors, cervical cancer, endometrial cancer, ovarian cancer, uterine sarcoma, vaginal cancer, female gonadal cancer, female urethral cancer or skin cancer. The blood cancer above is specifically exemplified by leukemia, malignant lymphoma, multiple myeloma or aplastic anemia.

Further, the photothermal composition of the present invention is characteristically used for the treatment of skin disease due to its antibacterial activity mediated by photothermal action. The skin disease above is specifically exemplified by acne, warts, atopy, eczema, lipomas, sebaceous cysts, epidermal cysts, epithelial cysts, subcutaneous cysts or skin fibrosis.

In addition, the photothermal composition of the present invention can be used for the improvement of skin absorption of a functional material for cosmetics. The functional material for cosmetics can be any liquid or solid substance having moisture containing, ultraviolet blocking, whitening, wrinkle reducing or irritation preventing functions.

The functional material above is exemplified by such extracts as avocado extract, fumitoli extract, carrot extract, Moutan cortex extract, Pueraria lobata root extract, Stone root extract, Lady's horsetail extract, lady mantil extract, horsetail extract, soybean embryo extract, wheat germ extract, radish extract, Laminaria japonica extract, Sanguisorba officinalis L. extract, Cinnamomum cassia bark extract, ginger extract, Ephedra distachya extract, herb extract, vitamin F and apple seed extract. The functional material can also be a substance containing components such as arbutin, ethylascorbyl ether, retinol, retinylpalmitate, adenosine, polyethoxylated retinamide, or a commercial product or a cosmetic ingredient for medicines containing them.

For example, the hyaluronic acid-gallic acid conjugate can be used for the purpose of promoting skin absorption of a functional substance by mixing with paste, gel, cream, lotion, powder, solid soap, wax, shampoo, rinse, solution, suspension, emulsion, mineral cosmetic, oil, emulsion foundation, soft lotion, nutritional lotion, nutritional cream, massage cream, essence, cleansing cream, cleansing foam, pack, pack base, eye cream, perfume, ointment, cleansing water, powder and spray, etc.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1: Preparation of Coordination Complex of Catechol and Iron Ions

5 mg/ml of catechol dissolved in TDW (triple distilled water) was mixed with 5 mg/ml of iron chloride dissolved in TDW at the ratio of 1:1 (v/v) to form coordination bonds, leading to the preparation of a coordination complex.

### Example 2: Preparation of Coordination Complex of Dopamine and Iron Ions

5 mg/ml of catechol amine dissolved in TDW (triple distilled water) was mixed with 5 mg/ml of iron chloride dissolved in TDW at the ratio of 1:1 (v/v) to form coordination bonds, leading to the preparation of a coordination complex.

### Example 3: Preparation of Coordination Complex of Epigallocatechin Gallate (EGCG) and Iron Ions

5 mg/ml of epigallocatechin gallate dissolved in TDW (triple distilled water) was mixed with 10 mg/ml of iron chloride dissolved in TDW at the ratio of 1:1 (v/v) to form coordination bonds, leading to the preparation of a coordination complex.

### Example 4: Preparation of Coordination Complex of Gallic Acid and Iron Ions

5 mg/ml of gallic acid dissolved in TDW (triple distilled water) was mixed with 5 mg/ml of iron chloride dissolved in TDW at the ratio of 1:1 (v/v) to form coordination bonds, leading to the preparation of a coordination complex.

### Example 5: Preparation of Coordination Complex of Tannic Acid and Iron Ions

5 mg/ml of tannic acid dissolved in TDW (triple distilled water) was mixed with 10 mg/ml of iron chloride dissolved in TDW at the ratio of 1:1 (v/v) to form coordination bonds, leading to the preparation of a coordination complex.

### Example 6: Formation of Hydrogel by Crosslinking Between Hyaluronic Acid-Gallic Acid Conjugate and Iron Ions

### Step 1: Synthesis of Hyaluronic Acid-Gallic Acid Conjugate (Gallic Acid-Conjugated Hyaluronic Acid)

100 g of hyaluronic acid was dissolved in 0.3 M NaHCO₃, to which 63 mg of EDC (1-Ethyl-3-(3-dimethylaminopropyl)carbodiimide) and 50 mg of NHS (N-hydroxysuccinimide) were added, followed by reaction for 3 hours.

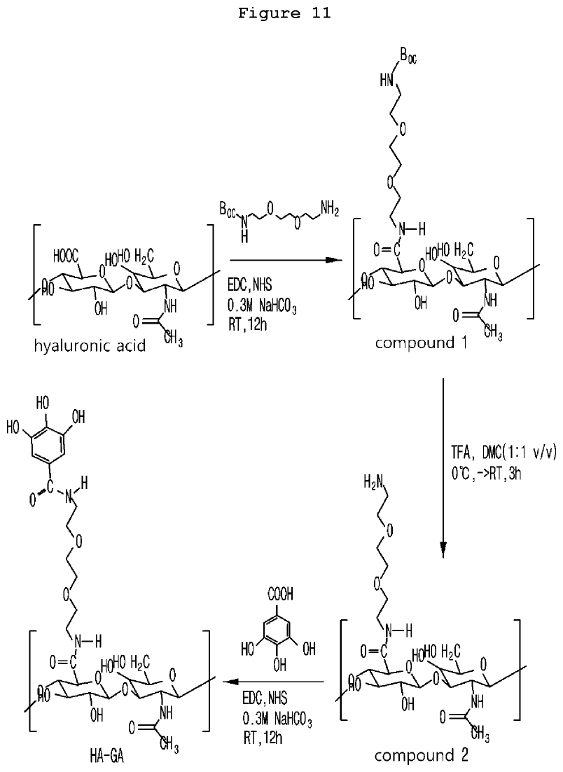

50μ [symbol] of N-Boc-2, 2-(ethylenedioxy) diethylamine was added to the mixture above, followed by reaction at room temperature for 12 hours (Compound 1, FIG. 11). Unreacted N-Boc-2,2′-(ethylenedioxy)diethylamine, EDC and NHS were eliminated by using a dialysis bag (molecular weight cutoff: 2000).

A mixture of 5 ml of TFA (trifluoroacetic acid) and 5 ml of DCM (dichloromethane) at the ratio of 1:1 was added thereto, followed by reaction at 0° C. for 3 hours (Compound 2, FIG. 11).

90 mg of gallic acid was dissolved in 0.3 M NaHCO₃, to which 126 mg of EDC and 100 mg of NHS were added, followed by reaction at room temperature for 3 hours. DCM and TFA were eliminated using a defreezer. The mixture above was mixed with Compound 2, followed by reaction for 12 hours. Unreacted gallic acid, EDC and NHS were eliminated by using a dialysis bag (molecular weight cutoff: 2000).

### Step 2: Formation of Hydrogel by Crosslinking by Iron Ion

Hyaluronic acid-gallic acid conjugate prepared in step 1 dissolved in PBS (phosphate buffered saline, 15 mg/ml) was mixed with iron chloride dissolved in PBS (phosphate buffered saline, 5 mg/ml) at the ratio of 1:1 (v/v). As soon as the two solutions were added, hydrogel was formed as they were mixed.

### Experimental Example 1: Photothermal Effect of Coordination Complex of Catechol and Iron Ions

<1-1> Experiment Method

50μ [symbol] of the catechol/iron ion coordination complex prepared in Example 1 was placed in an EP-tube. The tube was irradiated with 1.2 W 808 nm laser for 1 minute. Then, the temperature was measured using a thermal sensing camera.

<1-2> Experiment Result

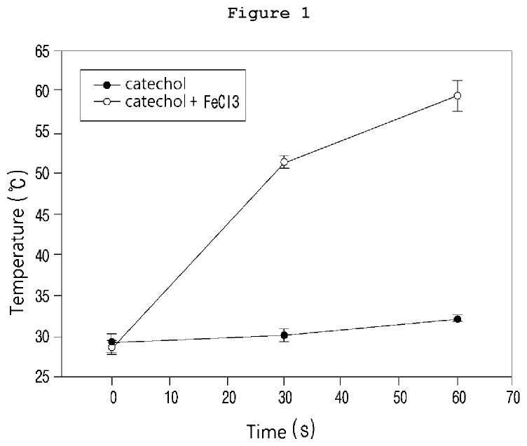

The results are shown in FIG. 1.

It was confirmed that the temperature of the catechol/iron ion coordination complex was raised to 60° C. or more.

### Experimental Example 2: Photothermal Effect of Coordination Complex of Dopamine and Iron Ions

<2-1> Experiment Method

50μ [symbol] of the dopamine/iron ion coordination complex prepared in Example 2 was placed in an EP-tube. The tube was irradiated with 1.2 W 808 nm laser for 1 minute. Then, the temperature was measured using a thermal sensing camera.

<2-2> Experiment Result

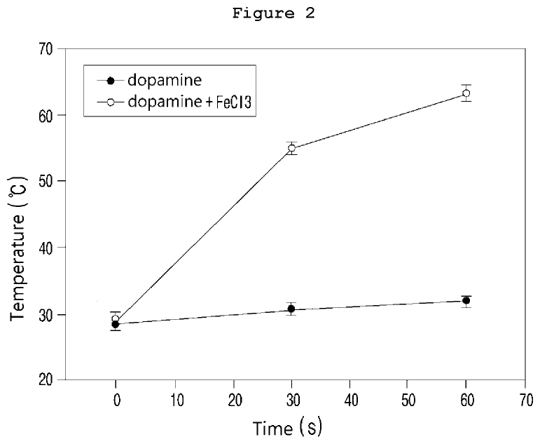

The results are shown in FIG. 2.

It was confirmed that the temperature of the catecholamine/iron ion coordination complex was raised to 60° C. or more.

### Experimental Example 3: Photothermal Effect of Coordination Complex of Epigallocatechin Gallate (EGCG) and Iron Ions

<3-1> Experiment Method

50μ [symbol] of the epigallocatechin gallate (EGCG)/iron ion coordination complex prepared in Example 3 was placed in an EP-tube. The tube was irradiated with 1.2 W 808 nm laser for 1 minute. Then, the temperature was measured using a thermal sensing camera.

<3-2> Experiment Result

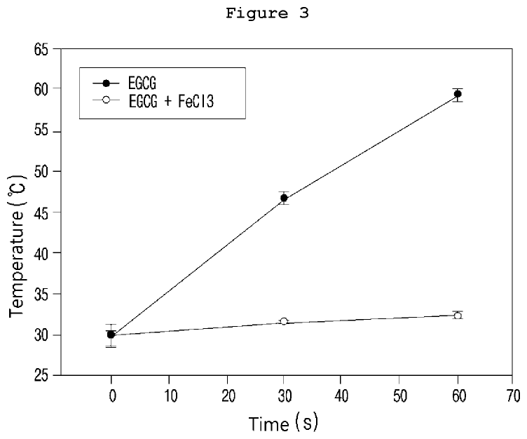

The results are shown in FIG. 3.

It was confirmed that the temperature of the epigallocatechin/iron ion coordination complex was raised to 60° C. or more.

### Experimental Example 4: Photothermal Effect of Coordination Complex of Gallic Acid and Iron Ions

<4-1> Experiment Method

50μ [symbol] of the gallic acid/iron ion coordination complex prepared in Example 4 was placed in an EP-tube. The tube was irradiated with 1.2 W 808 nm laser for 1 minute. Then, the temperature was measured using a thermal sensing camera.

<4-2> Experiment Result

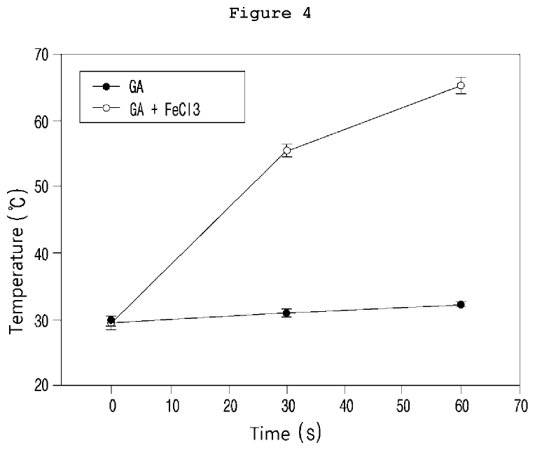

The results are shown in FIG. 4.

It was confirmed that the temperature of the gallic acid/iron ion coordination complex was raised to 60° C. or more.

### Experimental Example 5: Photothermal Effect of Coordination Complex of Tannic Acid and Iron Ions

<5-1> Experiment Method

50μ [symbol] of the tannic acid/iron ion coordination complex prepared in Example 5 was placed in an EP-tube. The tube was irradiated with 1.2 W 808 nm laser for 1 minute. Then, the temperature was measured using a thermal sensing camera.

<5-2> Experiment Result

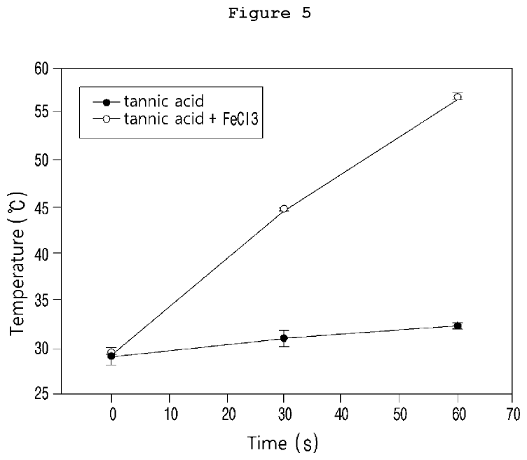

The results are shown in FIG. 5.

It was confirmed that the temperature of the tannic acid/iron ion coordination complex was raised to 60° C. or more.

### Experimental Example 6: Effect of Coordination Complex of Catechol and Iron Ions on Photothermal Therapy

<6-1> Experiment Method

Cancer cells (KB cells) were cultured in a 24 well plate, and the cultured cells were collected in an EP tube. Cell pellet was made using a centrifuge (1000 rpm, 3 minutes). 20μ [symbol] of the supernatant was left, to which 40μ [symbol] of the catechol/iron ion coordination complex prepared in Example 1 dissolved in PBS was added. The mixture was irradiated with 1.2 W 808 nm laser for 5 minutes. After removing the supernatant, 400μ [symbol] of medium was added thereto. The cells were cultured again in a 24 well plate for a day. The effect on photothermal therapy was measured by MTT assay.

<6-2> Experiment Result

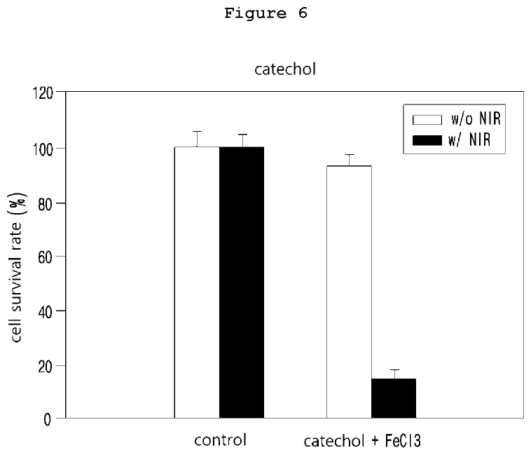

The results are shown in FIG. 6.

As a result, when the coordination complex was irradiated with laser, the cell viability was reduced to 20% or less.

### Experimental Example 7: Effect of Coordination Complex of Dopamine and Iron Ions on Photothermal Therapy

<7-1> Experiment Method

Cancer cells (KB cells) were cultured in a 24 well plate, and the cultured cells were collected in an EP tube. Cell pellet was made using a centrifuge (1000 rpm, 3 minutes). 20μ [symbol] of the supernatant was left, to which 40μ [symbol] of the dopamine/iron ion coordination complex prepared in Example 2 dissolved in PBS was added. The mixture was irradiated with 1.2 W 808 nm laser for 5 minutes. After removing the supernatant, 400μ [symbol] of medium was added thereto. The cells were cultured again in a 24 well plate for a day. The effect on photothermal therapy was measured by MTT assay.

<7-2> Experiment Result

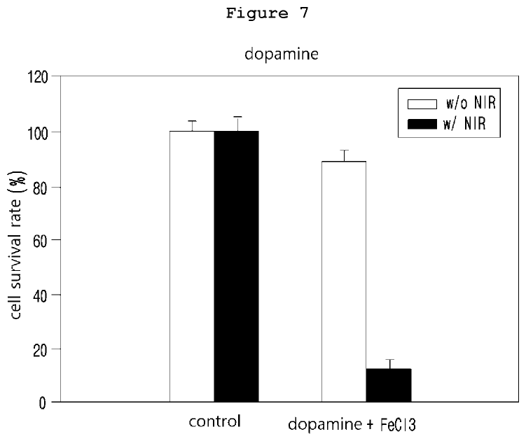

The results are shown in FIG. 7.

As a result, when the coordination complex was irradiated with laser, the cell viability was reduced to 20% or less.

### Experimental Example 8: Effect of Coordination Complex of Epigallocatechin Gallate (EGCG) and Iron Ions on Photothermal Therapy

<8-1> Experiment Method

Cancer cells (KB cells) were cultured in a 24 well plate, and the cultured cells were collected in an EP tube. Cell pellet was made using a centrifuge (1000 rpm, 3 minutes). 20μ [symbol] of the supernatant was left, to which 40μ [symbol] of the epigallocatechin gallate/iron ion coordination complex prepared in Example 3 dissolved in PBS was added. The mixture was irradiated with 1.2 W 808 nm laser for 5 minutes. After removing the supernatant, 400μ [symbol] of medium was added thereto. The cells were cultured again in a 24 well plate for a day. The effect on photothermal therapy was measured by MTT assay.

<8-2> Experiment Result

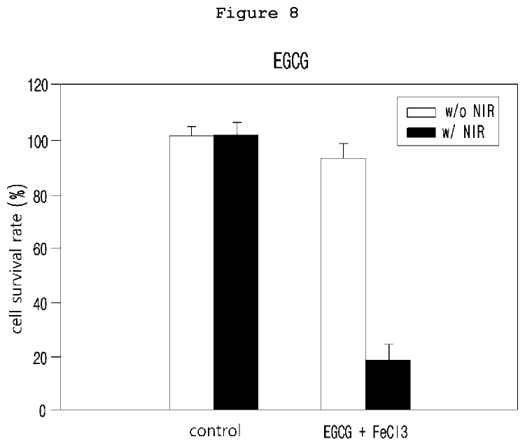

The results are shown in FIG. 8.

As a result, when the coordination complex was irradiated with laser, the cell viability was reduced to 20% or less.

### Experimental Example 9: Effect of Coordination Complex of Gallic Acid and Iron Ions on Photothermal Therapy

<9-1> Experiment Method

Cancer cells (KB cells) were cultured in a 24 well plate, and the cultured cells were collected in an EP tube. Cell pellet was made using a centrifuge (1000 rpm, 3 minutes). 20μ [symbol] of the supernatant was left, to which 40μ [symbol] of the gallic acid/iron ion coordination complex prepared in Example 4 dissolved in PBS was added. The mixture was irradiated with 1.2 W 808 nm laser for 5 minutes. After removing the supernatant, 400μ [symbol] of medium was added thereto. The cells were cultured again in a 24 well plate for a day. The effect on photothermal therapy was measured by MTT assay.

<9-2> Experiment Result

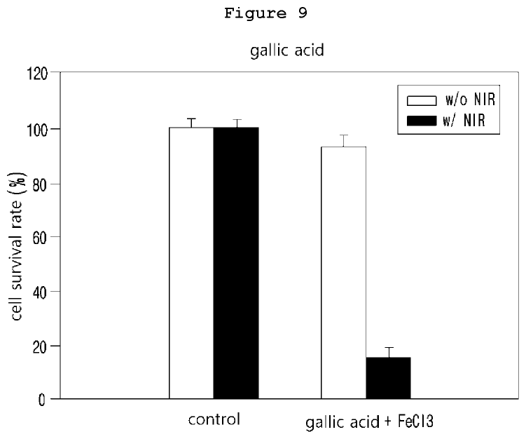

The results are shown in FIG. 9.

As a result, when the coordination complex was irradiated with laser, the cell viability was reduced to 20% or less.

### Experimental Example 10: Effect of Coordination Complex of Tannic Acid and Iron Ions on Photothermal Therapy

<10-1> Experiment Method

Cancer cells (KB cells) were cultured in a 24 well plate, and the cultured cells were collected in an EP tube. Cell pellet was made using a centrifuge (1000 rpm, 3 minutes). 20μ [symbol] of the supernatant was left, to which 40μ [symbol] of the tannic acid/iron ion coordination complex prepared in Example 5 dissolved in PBS was added. The mixture was irradiated with 1.2 W 808 nm laser for 5 minutes. After removing the supernatant, 400μ [symbol] of medium was added thereto. The cells were cultured again in a 24 well plate for a day. The effect on photothermal therapy was measured by MTT assay.

<10-2> Experiment Result

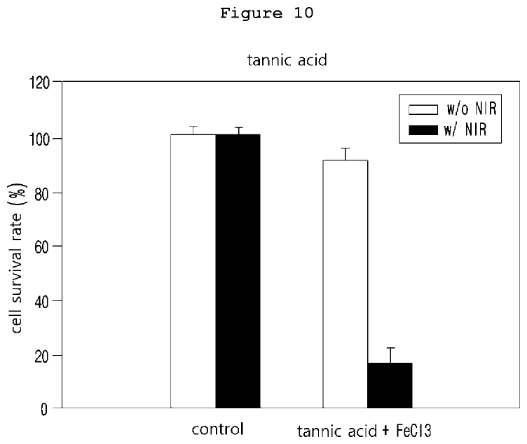

The results are shown in FIG. 10.

As a result, when the coordination complex was irradiated with laser, the cell viability was reduced to 20% or less.

### Experimental Example 11: Confirmation of Synthesis of Hyaluronic Acid-Gallic Acid Conjugate (Gallic Acid-Conjugated Hyaluronic Acid)

The hyaluronic acid-gallic acid conjugate synthesized in step 1 of Example 6 was dissolved in 1 m [symbol] of D₂O at the concentration of 5 mg/m [symbol] .

To confirm the synthesis of the hyaluronic acid-gallic acid conjugate, H NMR was analyzed up to 0-10 ppm by using 600 MHz NMR. As a result, hyaluronic acid peak was confirmed at 1.8-2.0 ppm, hydrogen peak was confirmed at 3.0-4.0 ppm, and gallic acid hydrogen (conjugate) peak was confirmed at 7.5 ppm. In addition, diethylamine hydrogen (linker) peak was confirmed at 2.8-2.9 ppm. From the above results, it was confirmed that the hyaluronic acid-gallic acid conjugate was successfully synthesized.

### Experimental Example 12: Evaluation of Swelling of Hydrogel Formed by Hyaluronic Acid-Gallic Acid Conjugate and Iron Ions

<12-1> Experiment Method

All moisture of the hydrogel formed in Example 6 was removed using a freeze-dryer and the weight of the dried hydrogel was measured.

TDW was added to the hydrogel, and the weight was measured at each time point using a balance. The temperature was fixed at 37° C. Swelling of the hydrogel was measured using the following equation. Swelling=(weight_{swelled} hydrogel-weight_{dried hydrogel})/weight_{dried hydrogel}×100%.

<12-2> Experiment Result

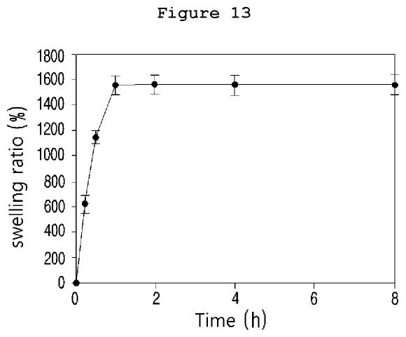

The results are shown in FIG. 13.

As a result, it was confirmed that swelling was increased over the time up to 1500%.

### Experimental Example 13: Evaluation of Viscosity and Viscoelasticity of Hydrogel Formed by Hyaluronic Acid-Gallic Acid Conjugate and Iron Ions

<13-1> Experiment Method

Viscosity and viscoelasticity of the hydrogel formed in Example 6 were measured using a rotational rheometer. Viscosity, loss modulus and storage modulus were measured in the range between 0.1 and 50 Hz.

<13-2> Experiment Result

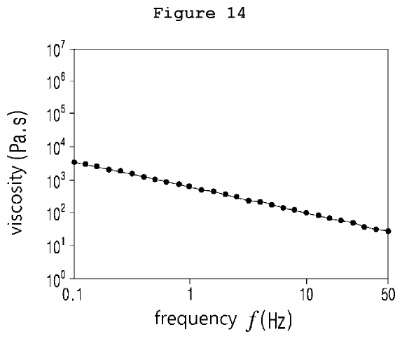

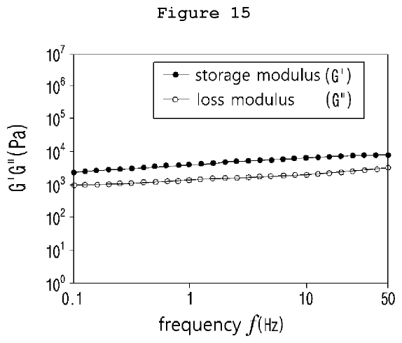

The results are shown in FIGS. 14 and 15.

As a result, it was confirmed that the hydrogel formed by hyaluronic acid-gallic acid conjugate and iron ions had viscosity and viscoelasticity.

### Experimental Example 14: Evaluation of Photothermal Effect of Hydrogel Formed by Hyaluronic Acid-Gallic Acid Conjugate and Iron Ions

<14-1> Experiment Method

The hydrogel formed in Example 6 was irradiated with 1.2 W 808 nm near infrared ray. Then, time dependent temperature changes were measured using a thermal imaging camera.

<14-2> Experiment Result

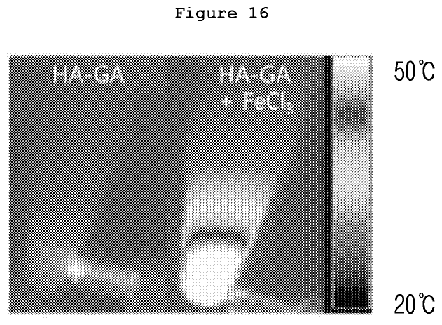

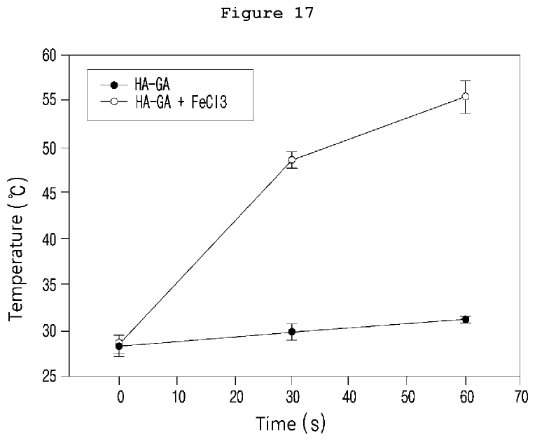

The results are shown in FIGS. 16 and 17.

As a result, when the formed hydrogel was irradiated with infrared laser, the temperature of the hydrogel was raised to 55° C. or more.

### Experimental Example 15: Confirmation of Antitumor Effect of Hydrogel Formed by Hyaluronic Acid-Gallic Acid Conjugate and Iron Ions

<15-1> Experiment Method

Cancer cells (KB cells) were cultured in a 24 well plate, and the cultured cells were collected in an Eppendorf tube.

Cancer cell pellet was made using a centrifuge (1000 rpm, 3 minutes). 40μ [symbol] of the supernatant was left, to which the hydrogel formed in Example 6 was added.

The mixture was irradiated with 1.2 W 808 nm laser for 5 minutes. After removing the supernatant, 400μ [symbol] of medium was added thereto. The cells were cultured again in a 24 well plate for a day. The effect on photothermal therapy was measured by MTT assay and live cell assay.

<15-2> Experiment Result

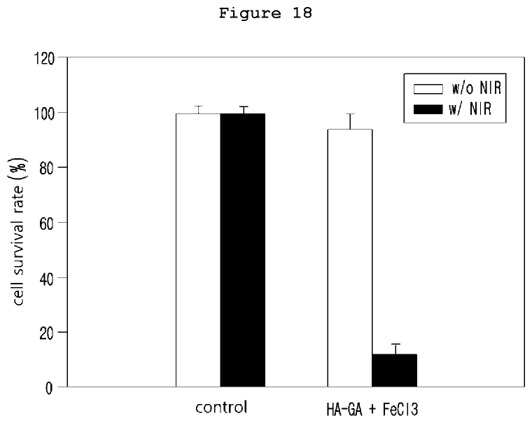

The results are shown in FIG. 18.

As a result, when the hydrogel was irradiated with laser, the cell viability was reduced to 20% or less.

### Experimental Example 16: Confirmation of Formation of Hydrogel Crosslinked by Hyaluronic Acid-Gallic Acid Conjugate and Iron Ions Under Mouse Subcutis

<16-1> Experiment Method

The hyaluronic acid-gallic acid conjugate prepared in step 1 of Example 6 dissolved in phosphate buffer (15 mg/ml) and iron chloride dissolved in phosphate buffer (5 mg/ml) were filled in two sections of a dual syringe, respectively, which was injected into the upper part of the right hind leg of Balb/c mouse. The mouse was then euthanized and the formation of hydrogel was confirmed using anatomical tools.

<16-2> Experiment Result

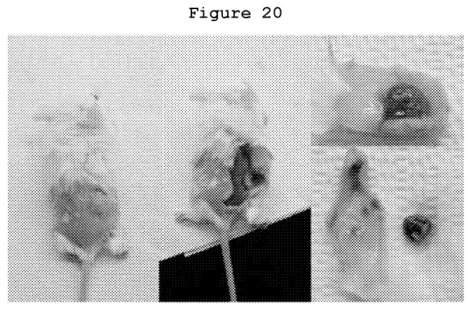

The results are shown in FIG. 20.

As a result, it was confirmed that hydrogel was formed well under the mouse subcutis.

### Experimental Example 17: Confirmation of Photothermal Effect of Hydrogel Formed by Hyaluronic Acid-Gallic Acid Conjugate and Iron Ions in Animal Model

<17-1> Experiment Method

The hyaluronic acid-gallic acid conjugate prepared in step 1 of Example 6 dissolved in phosphate buffer (15 mg/ml) and iron chloride dissolved in phosphate buffer (5 mg/ml) were filled in two sections of a dual syringe, respectively, which was injected into the upper part of the right hind leg of Balb/c mouse.

After the in vivo injection, the hyaluronic acid-gallic acid conjugate and iron chloride formed hydrogel therein. The hydrogel was irradiated with 1.2 W 808 nm near infrared ray for 1 minute, and then the temperature changes were measured using a thermal imaging camera.

As the control, the hyaluronic acid-gallic acid conjugate prepared in step 1 of Example 6 dissolved in phosphate buffer (15 mg/ml) was injected into the upper part of the right hind leg of Balb/c mouse, followed by measurement of the temperature changes by the same manner as described above.

<17-2> Experiment Result

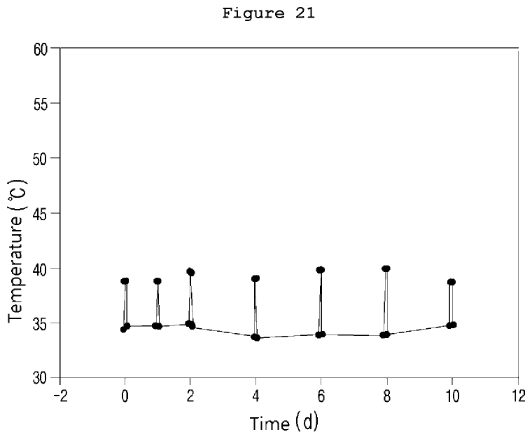

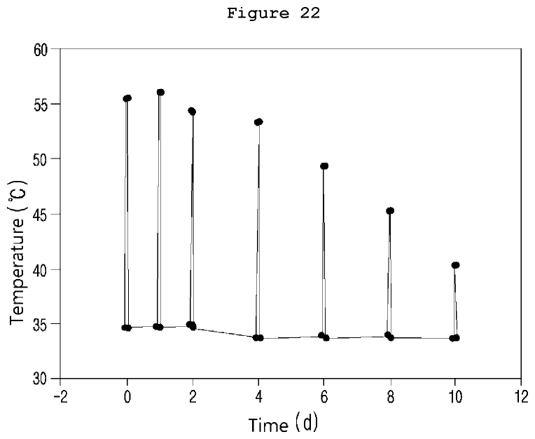

The results are shown in FIGS. 21 and 22.

As a result, compared with the control mouse subcutaneously injected with the hyaluronic acid-gallic acid conjugate alone, the photothermal effect was constantly observed in the experimental group mouse in which the hydrogel was formed.

Particularly, when near infrared ray was irradiated once a day for 4 days, the temperature was raised to 50° C. or more due to the sustainability of the hydrogel, confirming the photothermal effect.

### Experimental Example 18: Confirmation of Antitumor Effect of Hydrogel Formed by Hyaluronic Acid-Gallic Acid Conjugate and Iron Ions in Mice

<18-1> Experiment Method

A tumor was generated in the size of 100 mm³−200 mm³ by injecting 3×10⁶ cancer cells (KB cell) in the upper part of the right hind leg of Balb/c mouse.

The hyaluronic acid-gallic acid conjugate prepared in step 1 of Example 6 dissolved in phosphate buffer (15 mg/ml) and iron chloride dissolved in phosphate buffer (5 mg/ml) were filled in two sections of a dual syringe, respectively, followed by intra tumoral injection.

Then, the tumor was irradiated with 1.2 W 808 nm near infrared ray for 5 minutes and the tumor size was measured every 3 days.

<18-2> Experiment Result

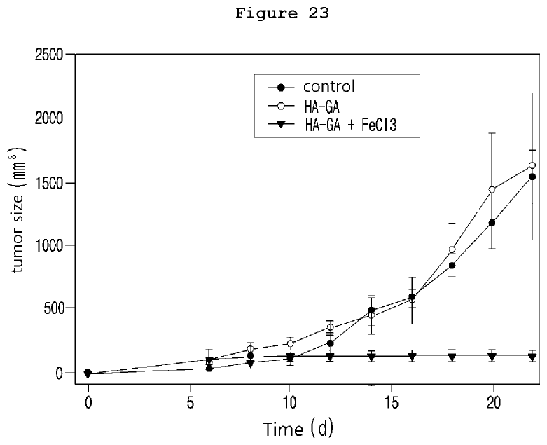

The results are shown in FIG. 23.

As a result, it was confirmed that the tumor size was suppressed by the effect of the hydrogel formed by hyaluronic acid-gallic acid conjugate and iron ions on photothermal therapy.

### Experimental Example 19: Preparation of Hydrogel and Gallic Acid/Iron Coordination Complex and Photothermal Effect

<19-1> Experiment Method

The gallic acid/iron coordination complex prepared in Example 4 was mixed with hydrogel at the ratio of 5:10 (w/w). The prepared hydrogel and gallic acid/iron coordination complex mixture was applied thinly, followed by irradiation with 808 nm near infrared ray at the intensity of 0.5-0.75 W from the distance of 1 cm, 2 cm and 4 cm, respectively.

<19-2> Experiment Result

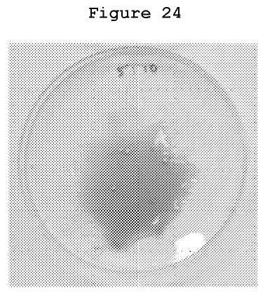

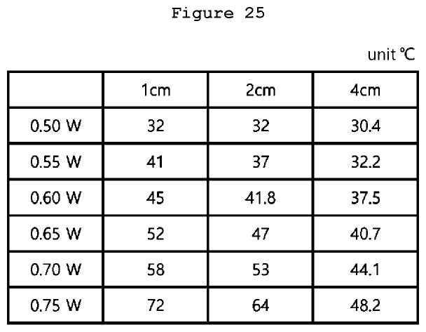

The results are shown in FIGS. 24 and 25.

### Experimental Example 20: Antibacterial Effect of Catechol Derivative/Iron Coordination Complex

<20-1> Experiment Method

E. coli (gram negative) and S. aureus (gram positive) were cultured and then diluted to optical density (OD) of 0.3. 100 μl of the diluted cells was loaded in an EP tube. Pellet was made using a centrifuge and the supernatant was removed. The coordination complex prepared in Example <2-5> was added thereto by 50μ [symbol] , followed by irradiation with 1.2 W 808 nm laser for 5 minutes. The mixture was plated on an agar plate, followed by incubation for 24 hours.

<20-2> Experiment Result

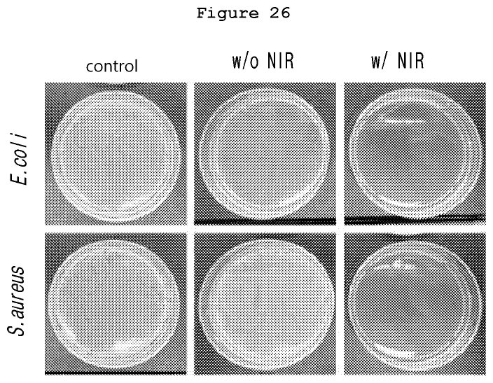

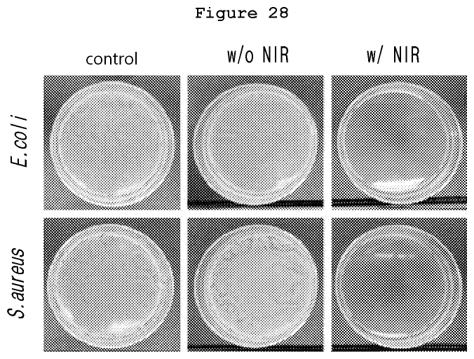

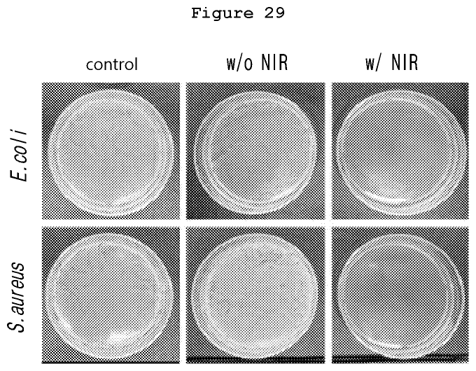

The results are shown in FIGS. 26, 28, 29 and 30.

### INDUSTRIAL APPLICABILITY

The composition of the present invention displays a remarkable effect on photothermal therapy since the temperature of the applied area can be raised at least 50° C. by near infrared ray irradiation, after the injection. The composition can be combined with a biocompatible material to have biocompatibility and can act selectively on a local site to minimize side effects. The composition also has an effect of continuous photothermal treatment because it is present in the administration site for a few days after injection. Therefore, the composition of the present invention can be used for anticancer treatment.

1. A method for treating cancer comprising:
administering a first composition comprising a metal salt and a second composition comprising a conjugate comprising a biocompatible material and a catechol derivative; and
irradiating near infrared,
wherein,
the first composition and the second composition are separately administered in a dual syringe;
the biocompatible material is hyaluronic acid; and
the catechol derivative is tannic acid or a compound represented by formula 1 below: 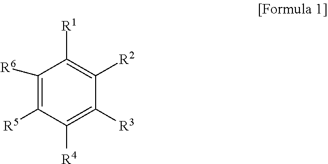
wherein,
R¹ and R² are —OH;
R³ is —H, or —OH;
R⁴ is —H, or ethyl amine;
R⁵ is —H, or —COOM,
herein M is —H, or epigallocatechinyl; and
R⁶ is —H,
the first composition and the second composition form a hydrogel by a coordination complex formed by the metal ion of the metal salt and the catechol derivative, and
when near infrared ray irradiates the hydrogel, a photothermal effect is induced to provide temperature higher than 50° C.

2. The method according to claim 1, wherein the metal salt is a transition metal salt.

3. The method according to claim 2, wherein the metal of the transition metal salt is iron (Fe).

4. The method according to claim 1, wherein the cancer is a solid cancer or a blood cancer.

5. The method according to claim 4, wherein the solid cancer is selected from the group consisting of brain tumor, benign astrocytoma, malignant astrocytoma, pituitary adenoma, meningioma, brain lymphoma, oligodendroglioma, intracranial carcinoma, ependymoma, brain stem tumor, head and neck cancer, laryngeal cancer, oropharyngeal cancer, nasal cavity cancer, nasopharyngeal cancer, salivary gland cancer, hypopharyngeal cancer, thyroid cancer, oral cancer, thoracic tumor, small cell lung cancer, non-small cell lung cancer, thymic carcinoma, mediastinal tumor, esophageal cancer, breast cancer, male breast cancer, abdominal tumor, stomach cancer, liver cancer, gallbladder cancer, bile duct cancer, pancreatic cancer, small bowel cancer, colon cancer, anal cancer, bladder cancer, kidney cancer, male genital tumor, penile cancer, prostate cancer, female genital tumors, cervical cancer, endometrial cancer, ovarian cancer, uterine sarcoma, vaginal cancer, female gonadal cancer, female urethral cancer and skin cancer.

6. The method according to claim 4, wherein the blood cancer is selected from the group consisting of leukemia, malignant lymphoma, multiple myeloma and aplastic anemia.
